(19) United States
(12) Patent Application Publication
(10) Pub. No.: US 2012/0095032 A1
(43) Pub. Date: Apr. 19, 2012

(54) METHODS FOR TREATING LEUKEMIA AND MYELODYSPLASTIC SYNDROME, AND METHODS FOR IDENTIFYING AGENTS FOR TREATING SAME

(75) Inventors: Tomas Mustelin, Seattle, WA (US); Lutz Tautz, San Diego, CA (US); Nicolas David Peter Cosford, San Diego, CA (US); Eduard Sergienko, San Diego, CA (US)

(73) Assignee: Sanford-Burnham Medical Research Institute

(21) Appl. No.: 13/312,391

(22) Filed: Dec. 6, 2011

Related U.S. Application Data
(63) Continuation of application No. 11/975,082, filed on Oct. 17, 2007.

(51) Int. Cl. A61K 31/437; C12N 5/10; C12N 5/071; A61P 35/02; A61P 7/00

(52) U.S. Cl. 514/293; 435/375

(57) ABSTRACT

The present disclosure relates to methods for treating leukemia, pre-leukemic conditions, as well as myelodysplastic syndrome and acute myelogenous leukemia. The present disclosure further relates to compounds that can be used for treating leukemia, pre-leukemic conditions, as well as myelodysplastic syndrome and acute myelogenous leukemia. The present disclosure also relates to methods for identifying compounds that can be used for treating leukemia, pre-leukemic conditions, as well as myelodysplastic syndrome.

### CROSS-REFERENCE TO RELATED APPLICATIONS

This application is a Continuation of U.S. application Ser. No. 11/975,082, filed Oct. 17, 2007. Application Ser. No. 11/975,082, filed Oct. 17, 2007, is hereby incorporated herein by reference in its entirety.

### STATEMENT REGARDING FEDERALLY SPONSORED RESEARCH

This invention was made with government support under Grant RFA 04-017 awarded by the National Institutes of Health. The government has certain rights in the invention.

### FIELD OF THE DISCLOSURE

The present disclosure relates to methods for treating leukemia, pre-leukemic conditions, as well as myelodysplastic syndrome and acute myelogenous leukemia. The present disclosure further relates to compounds that can be used for treating leukemia, pre-leukemic conditions, as well as myelodysplastic syndrome and acute myelogenous leukemia. The present disclosure also relates to methods for identifying compounds that can be used for treating leukemia, pre-leukemic conditions, as well as myelodysplastic syndrome.

### BACKGROUND

Tyrosine phosphorylation (Hunter, T. et al., (1980), Transforming gene production of Rous sarcoma virus phosphorylates tyrosine. Proc. Natl. Acad. Sci. USA 77, 1311-1315) is a key mechanism for signal transduction and the regulation of a broad set of physiological processes characteristic of multicellular organisms, such as integration of signal transduction pathways, decisions to proliferate, differentiate or die, activation of large gene transcription programs, cell motility and morphology, and the transport of molecules in or out of cells.

Protein tyrosine phosphatases (PTPs), working with protein tyrosine kinases (PTKs), control the phosphorylation state of many proteins in the signal transduction pathways. HePTP is a tyrosine phosphatase expressed in hematopoietic cells and regulates the MAP kinases Erk and p38. It has now been found that HePTP is often dysregulated in the preleukemic disorder myelodysplastic syndrome, as well as in acute myelogeneous leukemia.

HePTP (Zanke, B. et al., (1992) Cloning and expression of an inducible lymphoid-specific, protein tyrosine phosphatase (HePTPase). Eur. J. Immunol. 22, 235-239; Adachi, M. et al., (1992) Molecular cloning and chromosomal mapping of a human protein-tyrosine phosphatase LC-PTP. Biochem. Biophys. Res. Commun. 186, 1607-1615) is a 38-kDa enzyme, which consists mainly of a PTP domain with only a short (approximately 50 residues) N-terminal extension. HePTP is expressed in bone marrow, thymus, spleen, lymph nodes, and all myeloid and lymphoid lineages and cell lines (Zanke, ibid.; Adachi, ibid.; Gjörloff-Wingren, A. et al., (2000) Subcellular localization of intracellular protein tyrosine phosphatases in T cells. Eur. J. Immunol. 30, 3412-2421).

The HePTP gene is located on chromosome1q32 (Zanke, B. et al., (1994) A hematopoietic protein tyrosine phosphatases (HePTP) gene that is amplified and over expressed in myeloid malignancies maps to chromosome 1q32.1. Leukemia 8, 236-244), which is often found in extra copies (usually partial trisomy) in bone marrow cells from patients with myelodysplastic syndrome, and acute myelogeneous leukemia(Fonatsch, C. et al., (1991) Partial trisomy 1q. A nonrandom primary chromosomal abnormality in myelodysplastic syndromes? Cancer Genet. Cytogenet. 56, 243-253; Manmaev, N. et al., (1988) Combined trisomy 1q and monosomy 17p due to translocation t(1:17) in a patient with melodysplastic syndrome. Cancer Genet. Cytogenet. 35, 21-25), which is characterized by disturbed hematopoiesis and an increased risk of acute leukemia. Amplification and over expression of HePTP was reported in a case of acute myelogenous leukemia (Zanke, B. et al., Leukemia 8, 236-244). Conversely, deletions of 1q32 have been reported in non-Hodgkin lymphomas and chronic lymphoproliferative disorders (Mitelman, F. et al., (1990) Report of the committee on chromosome changes n neoplasia. Cytogenet. Cell. Genet. 55, 358-86). These findings suggest that excess HePTP can correlate with reduced proliferation (in myelodysplastic syndrome) and loss of HePTP with increased cell proliferation and/or survival. A connection with proliferation is also supported by the finding that the HePTP gene is transcriptionally activated in T cell treated with IL-2 (Zanke, B. et al., (1992) Cloning and expression of an inducible lymphoid-specific, protein tyrosine phosphatase (HePTPase). Eur. J. Immunol. 22, 235-239; Adachi, M. et al., (1994) Induction of protein-tyrosine phosphatase LC-PTP by IL-2 in human T cells. Febs Lett. 338, 47-52). Although mRNA levels also increased several fold upon stimulation of normal mouse lymphocytes with phytohemagglutinin, lipopolysaccharide, Concanavalin A or anti-CD3, the HePTP protein was present in resting cells and its amount increased only moderately.

There is therefore a long felt need for compounds that can regulate the activity of HePTP or completely inhibit the activity of HePTP. These compounds can be used to treat patients having leukemia, pre-leukemic conditions, as well as other conditions including myelodysplastic syndrome and acute myelogenous leukemia.

### SUMMARY

The present disclosure relates to small molecule inhibitors of HePTP, pharmaceutical compositions comprising HePTP inhibitors, methods for inhibiting or controlling the activity of HePTP either in vivo, in vitro, or ex vivo, and to methods for treating humans having leukemia, pre-leukemic conditions, as well as myelodysplastic syndrome and acute myelogenous leukemia. Also disclosed is a method for determining whether a compound is an inhibitor of HePTP.

### DETAILED DISCLOSURE

In this specification and in the claims that follow, reference will be made to a number of terms, which shall be defined to have the following meanings:

By “pharmaceutically acceptable” is meant a material that is not biologically, clinically or otherwise undesirable, i.e., the material can be administered to an individual along with the relevant active compound without causing clinically unacceptable biological effects or interacting in a deleterious manner with any of the other components of the pharmaceutical composition in which it is contained.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

By the term “effective amount” of a compound as provided herein is meant a sufficient amount of the compound to provide the desired regulation of a desired function, such as gene expression, protein function, or a disease condition. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

The term “organic radical” defines a carbon containing moiety that forms a portion of a larger molecule, i.e. a moiety comprising at least one carbon atom, and can also often contain hydrogen atoms. Examples of organic radicals that comprises no heteroatoms are alkyls such as methyl, ethyl, n-propyl, or isopropyl moieties, or cyclic organic radicals such as phenyl or tolyl moieties, or 5,6,7,8-tetrahydro-2-naphthyl moieties. Organic radicals can and often do, however, optionally contain various heteroatoms such as halogens, oxygen, nitrogen, sulfur, phosphorus, or the like. Examples of organic residues include alkoxy or substituted alkoxy moieties such as methoxyl moieties or hydroxymethyl moieties, or in other examples triflouromethyl moieties, mono or di-methyl amino moieties, carboxy moieties, formyl moieties, amide moieties, etc. An organic radical can have, for example, 1-26 carbon atoms, 1-18 carbon atoms, 1-12 carbon atoms, 1-8 carbon atoms, or 1-4 carbon atoms. Organic radicals often have a hydrogen bound to at least some of the carbon atoms of the organic radical. In some embodiments, an organic radical can contain 1-10, or 1-5 heteroatoms bound thereto

The term “alkyl” denotes a hydrocarbon group or residue which is structurally similar to an alkane compound modified by the removal of one hydrogen from the non-cyclic alkane and the substitution therefore of a non-hydrogen moiety. “Normal” or

“Branched” alkyls comprise a non-cyclic, saturated, straight or branched chain hydrocarbon moiety having from 1 to 12 carbons, or 1 to 8 carbons, 1 to 6, or 1 to 4 carbon atoms. Examples of such alkyl radicals include methyl, ethyl, n-propyl, iso-propyl, n-butyl, sec-butyl, t-butyl, amyl, t-amyl, n-pentyl and the like. Lower alkyls comprise a noncyclic, saturated, straight or branched chain hydrocarbon residue having from 1 to 4 carbon atoms, i.e., C₁-C₄ alkyl.

The term “substituted alkyl” denotes an alkyl radical analogous to the above definition that is further substituted with one, two, or more additional organic or inorganic substituent groups. Suitable substituent groups include but are not limited to hydroxyl, cycloalkyl, amino, mono-substituted amino, di-substituted amino, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. When more than one substituent group is present then they can be the same or different. The organic substituent moieties can comprise from 1 to 12 carbon atoms, or from 1 to 6 carbon atoms, or from 1 to 4 carbon atoms.

The term “alkenyl” denotes an alkyl residue as defined above that also comprises at least one carbon-carbon double bond. Examples include but are not limited to vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2-heptenyl, 3-heptenyl, 4-heptenyl, 5-heptenyl, 6-heptenyl and the like. The term “alkenyl” includes dienes and trienes of straight and branch chains.

The term “substituted alkenyl” denotes an alkenyl residue, as defined above that is substituted with one or more additional moieties, but preferably one, two or three groups, selected from halogen, hydroxyl, cycloalkyl, amino, mono-substituted amino, di-substituted amino, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy. When more than one group is present then they can be the same or different. The organic substituent groups can comprise from 1 to 12 carbon atoms, or from 1 to 6 carbon atoms, or from 1 to 4 carbon atoms.

The term “alkynyl” denotes a residue as defined above that comprises at least one carbon-carbon double bond. Examples include but are not limited ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

The term “cycloalkyl” denotes a hydrocarbon group or residue which is structurally similar to a cyclic alkane compound modified by the removal of one hydrogen from the cyclic alkane and substitution therefore of a non-hydrogen moiety. Cycloalkyls typically comprise a cyclic radical containing 3 to 8 ring carbons, such as cyclopropyl, cyclobutyl, cyclopentyl, cyclopenyl, cyclohexyl, cycloheptyl and the like. Cycloalkyl radicals can be multicyclic and can contain a total of 3 to 18 carbons, or preferably 4 to 12 carbons, or 5 to 8 carbons. Examples of multicyclic cycloalkyls include decahydronapthyl, adamantyl, and like radicals.

The term “substituted cycloalkyl” denotes a cycloalkyl residue as defined above that is further substituted with one, two, or more additional organic or inorganic groups that can include but are not limited to halogen, alkyl, substituted alkyl, hydroxyl, alkoxy, substituted alkoxy, carboxy, carboalkoxy, alkylcarboxamido, substituted alkylcarboxamido, dialkylcarboxamido, substituted dialkylcarboxamido, amino, mono-substituted amino or di-substituted amino. When the cycloalkyl is substituted with more than one substituent group, they can be the same or different. The organic substituent groups can comprise from 1 to 12 carbon atoms, or from 1 to 6 carbon atoms, or from 1 to 4 carbon atoms.

The term “cycloalkenyl” denotes a cycloalkyl radical as defined above that comprises at least one carbon-carbon double bond. Examples include but are not limited to cyclopropenyl, 1-cyclobutenyl, 2-cyclobutenyl, 1-cyclopentenyl, 2-cyclopentenyl, 3-cyclopentenyl, 1-cyclohexyl, 2-cyclohexyl, 3-cyclohexyl and the like. The term “substituted cycloalkenyl” denotes a cycloalkyl as defined above further substituted with one or more groups selected from halogen, alkyl, hydroxyl, alkoxy, substituted alkoxy, haloalkoxy, carboxy, carboalkoxy, alkylcarboxamido, substituted alkylcarboxamido, dialkylcarboxamido, substituted dialkylcarboxamido, amino, mono-substituted amino or di-substituted amino. When the cycloalkenyl is substituted with more than one group, they can be the same or different. The organic substituent groups can comprise from 1 to 12 carbon atoms, or from 1 to 6 carbon atoms, or from 1 to 4 carbon atoms.

The term “alkoxy” as used herein denotes an alkyl residue, as defined above, bonded directly to an oxygen atom, which is then bonded to another moiety. Examples include methoxy, ethoxy, n-propoxy, iso-propoxy, n-butoxy, t-butoxy, iso-butoxy and the like.

The term “substituted alkoxy” denotes an alkoxy residue of the above definition that is substituted with one or more substituent groups, but preferably one or two groups, which include but are not limited to hydroxyl, cycloalkyl, amino, mono-substituted amino, di-substituted amino, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy. When more than one group is present then they can be the same or different. The organic substituent groups can comprise from 1 to 12 carbon atoms, or from 1 to 6 carbon atoms, or from 1 to 4 carbon atoms.

The term “mono-substituted amino” denotes a moiety comprising an NH radical substituted with one organic substituent group, which include but are not limited to alkyls, substituted alkyls, cycloalkyls, aryls, or arylalkyls. Examples of mono-substituted amino groups include methylamino (—NH—CH₃); ethylamino (—NHCH₂CH₃), hydroxyethylamino (—NH—CH₂CH₂OH), and the like.

The term “di-substituted amino” denotes a moiety comprising a nitrogen atom substituted with two organic radicals that can be the same or different, which can be selected from but are not limited to aryl, substituted aryl, alkyl, substituted alkyl or arylalkyl, wherein the terms have the same definitions found throughout. Some examples include dimethylamino, methylethylamino, diethylamino and the like.

The term “haloalkyl” denotes an alkyl residue as defined above, substituted with one or more halogens, preferably fluorine, such as a trifluoromethyl, pentafluoroethyl and the like.

The term “haloalkoxy” denotes a haloalkyl residue as defined above that is directly attached to an oxygen to form trifluoromethoxy, pentafluoroethoxy and the like.

The term “acyl” denotes a R—C(O)— residue having an R group containing 1 to 8 carbons. Examples include but are not limited to formyl, acetyl, propionyl, butanoyl, iso-butanoyl, pentanoyl, hexanoyl, heptanoyl, benzoyl and the like, and natural or un-natural amino acids.

The term “acyloxy” denotes an acyl radical as defined above directly attached to an oxygen to form an R—C(O)O— residue. Examples include but are not limited to acetyloxy, propionyloxy, butanoyloxy, iso-butanoyloxy, benzoyloxy and the like.

The term “aryl” denotes a ring radical containing 6 to 18 carbons, or preferably 6 to 12 carbons, comprising at least one six-membered aromatic “benzene” residue therein. Examples of such aryl radicals include phenyl, naphthyl, and ischroman radicals. The term “substituted aryl” denotes an aryl ring radical as defined above that is substituted with one or more, preferably 1, 2, or 3 organic or inorganic substituent groups, which include but are not limited to a halogen, alkyl, substituted alkyl, hydroxyl, cycloalkyl, amino, mono-substituted amino, di-substituted amino, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, substituted heterocyclic ring wherein the terms are defined herein. The organic substituent groups can comprise from 1 to 12 carbon atoms, or from 1 to 6 carbon atoms, or from 1 to 4 carbon atoms.

The term “heteroaryl” denotes an aryl ring radical as defined above, wherein at least one of the ring carbons, or preferably 1, 2, or 3 carbons of the aryl aromatic ring has been replaced with a heteroatom, which include but are not limited to nitrogen, oxygen, and sulfur atoms. Examples of heteroaryl residues include pyridyl, bipyridyl, furanyl, and thiofuranyl residues. Substituted “heteroaryl” residues can have one or more organic or inorganic substituent groups, or preferably 1, 2, or 3 such groups, as referred to herein-above for aryl groups, bound to the carbon atoms of the heteroaromatic rings. The organic substituent groups can comprise from 1 to 12 carbon atoms, or from 1 to 6 carbon atoms, or from 1 to 4 carbon atoms.

The term “halo” or “halogen” refers to a fluoro, chloro, bromo or iodo group.

For the purposes of the present disclosure the terms “compound,” “analog,” and “composition of matter” stand equally well for the chemical entities described herein, including all enantiomeric forms, diastereomeric forms, salts, and the like, and the terms “compound,” “analog,” and “composition of matter” are used interchangeably throughout the present specification.

Compounds obtained from the National Institute of Health (NIH) Molecular Libraries Screening Centers Network were obtained and tested to determine if this library contained compounds that are inhibitors of HePTP and, therefore, compounds that can be used for inhibiting or controlling the activity of HePTP in vivo, in vitro, or ex vivo. The compounds of the present disclosure can also be used to treat a disease characterized by increased levels of HePTP activity. In addition, the compounds can be used for methods of controlling, treating, or mediating leukemia, pre-leukemic conditions, as well as myelodysplastic syndrome or acute myelogenous leukemia.

An assay has been developed that identifies compositions of matter that can inhibit HePTP and therefore serve as a method for controlling hematopoietic malignancies. The following is a description of a colorimetric HTS that can identify HePTP inhibitors.

### HTS ASSAY

The following assay can be conducted manually or can be carried out by a robotic station. The following solutions are prepared:
Solution 1: comprises 50 mM Bis-Tris, pH 6.0, 2.5 mM dithiothreitol (DTT), 0.0125% Tween™ 20.
Solution 2: comprises 6.875 nM HePTP in Solution 1.
Solution 3: comprises 1 mM p-nitrophenyl phosphate in membrane filtered water.
Solution 4: comprises 45 mM Na₃VO₄ in dimethylsufoxide (DMSO)/water (1:9).
Solution 5: comprises commercially available Biomol Green™ reagent.

For this assay, HePTP catalyzes the following reaction:

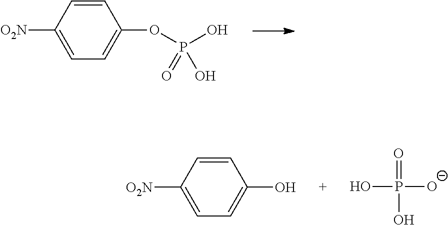

wherein p-nitrophenyl phosphate serves as a substrate and is cleaved to form p-nitrophenol and phosphate. The liberated phosphate complexes with the Biomol Green™ reagent to provide a quantitative colorimetric value for the amount of substrate cleaved.

HePTP HTS protocol:
1) 4 μL of 100 μM compounds to be tested in 10% DMSO were dispensed in columns 3-24 of Greiner 384-well clear microtiter plates (781101).
2) 4 μL of the following solutions are using the Multidrop bulk dispenser (Thermo):
   a) 10% DMSO column 1 (negative control)
   b) 45 mM Na₃VO₄ in 10% DMSO—column 2 (positive control).
3) 8 μL of HePTP working solution is added to the whole plate using WellMate bulk dispenser (Matrix).
4) 8 μL ofp-nitrophenyl phosphate working solution is added to the whole plate using WellMate bulk dispenser (Matrix).
5) Final concentrations of the components in the assay are as follows:
   a) 20 mM Bis-Tris, pH 6.0, 1.0 mM DTT, 0.005% Tween 20.
   b) 2.75 nM HePTP (columns 1-24
   c) 0.4 mM pNPP (columns 1-24)
   d) 9 mM Na₃VO₄ (column 2)
   e) 2% DMSO (columns 1-24)
   f) 20 μM compounds (columns 3-24)
6) Plates were incubated for 1 hour at room temperature.
7) 40 μL of Biomol Green™ reagent is added to the entire plate using the WellMate bulk dispenser (Matrix).
8) Plates are incubated for 30 minutes at room temperature
9) Absorbance at 620 nM is measured on the Envision plate reader (PerkinElmer).
10) Data analysis is performed using CBIS software (ChemInnovations, Inc). HePTP dose-response confirmation screening protocol:
1) Dose-response curves typically contain 10 concentrations of compounds obtained using 2-fold serial dilution. Compounds are then serially diluted in 100% DMSO, and then diluted with water to 10% final DMSO concentration. 4 μL of the selected compound in 10% DMSO are transferred into columns 3-22 of Greiner 384-well white small-volume plates (784075). Columns 1-2 and 23-24 contain 4 μL of Solution 4 and 10% DMSO, respectively.
2) 8 μL of Solution 2 is added to the whole plate using WellMate bulk dispenser (Matrix).
3) 8 μL of Solution 3 is added to the whole plate using WellMate bulk dispenser (Matrix).
4) Plates are incubated for 1 hour at room temperature.
5) Absorbance at 620 nM is then measured on the Envision plate reader (PerkinElmer).
6) Data analysis can be performed using and analysis software, inter alia, CBIS software (ChemInnovations, Inc) using sigmoidal dose-response equation through non-linear regression.

Compounds with greater than 50% inhibition of HePTP at 20-μM concentration are defined herein as actives of the primary screening. The primary screening actives can then proceed to the dose-response confirmation stage. Compounds that demonstrate IC₅₀ values in the range of analyzed concentrations are considered to be active as inhibitors of HePTP. Compounds that fail dose-response confirmation are assigned IC₅₀ values equal to 999 (μM) and are not considered to be inhibitors of HePTP according to the present disclosure.

To simplify the distinction between a compound that is inactive against inhibition of HePTP and compound that exhibit activity in the primary screen and in the confirmatory screening stage, a Tiered Activity Scoring System has been developed and is disclosed herein. The following is a description of the activity scoring for this disclosed HePTP.

### Activity Scoring

Activity scoring rules have been developed to consider various factors important in identifying HePTP inhibitors. Those factors include considerations such as:
a) compound efficacy;
b) potential interference of the compound with the assay; and
c) potential interference taking into account the screening stage at which relevant data is obtained.

The following is an outline of a scoring system for determining the results of testing in the HePTP assay disclosed herein:

1) First tier results are assigned a score, Score(1), from 0 to 40 and are reserved for the primary screening results. The data obtained are then correlated with the percent displacement in the assay that is demonstrated by a compound at 20 μM concentration and are assigned scores according to the following criteria:
a) If the primary % inhibition is less than 0%, then the results are assigned a score of 0;
b) If the primary % inhibition is greater than 100%, then the results are assigned a score of 40;
c) If the primary % inhibition is between 0% and 100%, then a the score is calculated by the formula:

Score(1)=(% Inhibition)×0.4

2) Second tier results are given a score, Score(2), from 41 to 80 and are assigned from dose-response confirmation data. These scores are assigned according to the following criteria:
a) Compounds that are found to be inactive in the confirmatory stage are assigned a score value equal 41.
b) Compounds having Scores assigned to compounds are linearly correlated to the compound's potency using the following equation

Score(2)=44+[6×(pIC₅₀−3]

wherein pIC₅₀ is defined herein as the negative log(10) of the IC₅₀ value as expressed in concentration units of mole/L. Compounds exhibiting an IC₅₀ of greater than 100 μM will have Score(2) values above 50.

3) Compounds having activity in the range of from 81 to 100 are designated as compounds that are inhibitors of HePTP.

### METHOD

The present disclosure relates to assays that provide a method for identifying an agent that inhibits the catalytic activity of HePTP, the assay comprising:
a) providing, in a first cocktail comprising, an amount of HePTP, an amount of a substrate capable of being dephosphorylated by HePTP, and thereby form phosphate, and an amount of a test agent, in an assay buffer comprising a reducing agent;
b) incubating said first cocktail under conditions suitable to allow for a substantial amount of dephosphorylation of said substrate by HePTP;
c) terminating the incubation; and
d) quantitating the amount of dephosphorylation in the first cocktail by comparing the amount of dephosphorylation in the first cocktail with the amount of dephosphorylation in a control comprising all of the ingredients of said first cocktail except for said test agent.

The following is a further example of an assay that provides a method for identifying an agent that inhibits the catalytic activity of HePTP, the assay comprising:
a) combining, in a first cocktail, an amount of HePTP, an amount of a substrate capable of being dephosphorylated by HePTP, and an amount of a test agent, in an assay buffer;
b) preparing a second cocktail that is a control, comprising all of the ingredients of said first cocktail except for said test agent;
c) incubating said first and second cocktails under conditions suitable to allow for a substantial amount of dephosphorylation of said substrate by HePTP;
d) terminating the incubation;
e) quantitating the amount of dephosphorylation in each of the cocktails; and
f) comparing said amounts of dephosphorylation; where a HePTP inhibitor is a test agent whose presence results in less dephosphorylation than its absence.

In one example of the disclosed method for identifying an HePTP inhibitor, the first cocktail comprises the isolated HePTP enzyme, p-nitrophenyl phosphate, and a compound that is the test target. The second cocktail comprises the isolated HePTP enzyme and p-nitrophenyl phosphate. The cocktails can further comprise an assay buffer comprising one or more adjunct ingredients to promote the stabilization of the enzyme and facilitate proper enzyme activity. A non-limiting example of an assay buffer suitable for use in promoting the stabilization of the enzyme and/or facilitate proper enzyme activity is Solution 1 disclosed herein above.

Step (a)

Step (a) comprises combining, in a first cocktail, an amount of HePTP, an amount of a substrate capable of being dephosphorylated by HePTP, and an amount of a test agent, in an assay buffer. The compound to be tested as an inhibitor of HePTP is added to a solution of the enzyme and substrate.

Control

A control, or second cocktail, is used as a reference point to compare the activity measured in the first cocktail described herein in step (a). The control comprises an amount of HePTP, an amount of a substrate capable of being dephosphorylated by HePTP, in an assay buffer. The control can be run concurrent with step (a) or the value or values of the control can be run periodically and used as a reference by which to compare the values resulting from testing an inhibitor in step (a).

Any suitable substrate capable of being dephosphorylated by HePTP can be used in the screening methods of the present invention. As those skilled in the art will appreciate, either the phosphate group(s) released from the substrate or the extent of phosphate remaining on a substrate containing the phosphate residue(s) can be measured as a readout of the extent of dephosphorylation by HePTP in the absence and presence of the test agent, as the case may be, depending upon what type of substrate is employed. In addition, the substrate may be bound to the reaction vessel using conventional methods, e.g., the substrate is a biotinylated form of a peptide having a phosphotyrosine residue and the walls of the reaction vessel are coated with streptavidin, which would then enable the use of a detection reagent, e.g., an anti-phosphotyrosine antibody, to quantitate the number of phosphotyrosine residues remaining after the dephosphorylation reaction is terminated, e.g., by rinsing the enzyme and the reaction products out of the reaction vessel, e.g., using a suitable buffer such as the assay buffer selected for the reaction. As described herein above a suitable substrate that is capable of being dephosphorylated is p-nitrophenyl phosphate.

The first and second cocktails can comprise any suitable reagents and conditions can be used in the assays of the present disclosure, and those skilled in the art will understand based on the present description and examples provided herein, how to select such conditions, depending upon the specific reagents employed and desired result sought. Any suitable buffer known by those skilled in the art which permits dephosphorylation reactions can be used in the present assays. Suitable buffers would include those which comprise a buffer, e.g., 10 mM Tris or Hepes, a salt, e.g., 150 mM NaCl, and a detergent, e.g., 0.05% Tween-20. For example, in an embodiment where Malachite Green is used as the readout, the enzyme buffer comprises 50 mM Tris, 0.15 M NaCl, 5 mM DTT, and 0.1% BSA. In addition, as those skilled in the art will appreciate, the enzyme is stabilized (from degradation) by the inclusion of a suitable reducing, e.g., DTT or BME. In one example, the reducing agent is present at a final concentration of from 1 mM to about 50 mM. In a further example the final concentration is 5 mM. In one example, Solution 1 that comprises 50 mM Bis-Tris, pH 6.0, 2.5 mM dithiothreitol (DTT), 0.0125% Tween™ 20 is suitable for use in the first and second cocktails of the presently disclosed assay.

Step (b)

Step (b) comprises incubating said first and second cocktails under conditions suitable to allow for a substantial amount of dephosphorylation of said substrate by HePTP. The purpose of this step is to allow the dephosphorylation reaction to proceed.

Those skilled in the art will also understand how to optimize any given assay in terms of the pH of the dephosphorylation reaction; however, as a general guide which should be suitable for most, if not all, reactions, depending, once again on the choice of reagents, the amounts of those reagents, and the desired result, a suitable pH range would be from about pH 5.0 to about pH 8.0, with a pH of about 7.4 being generally most preferred. Those skilled in the art would also know from conventional methods which reagents to use to adjust the pH in any given direction to avoid any unwanted interference with the dephosphorylation reaction.

Step (c)

Step (c) of the presently disclosed assay encompasses terminating the dephosphorylation reaction. One example of a method for terminating the reaction is to add an inhibitor, inter alia, Na₃VO₄. Another example for terminating the reaction is to add a reagent that terminates the dephosphorylation reaction and in addition provides a measure of the degree of dephosphorylation. Biomol Green™ can be used to both inhibit further dephosphorylation and to provide a colorimetric measure of the amount of dephosphorylation. When a reagent such as Biomol Green™ is used the measurement of the Biomol Green™ reagent/phosphate complex can be step (e) of the disclosed process.

Any suitable period of time can be selected for the dephosphorylation reaction such as, for example, a period of time from about 5 minutes to about 90 minutes, e.g., 30 minutes. Those skilled in the art will understand from conventional methods how to determine or “titrate” the period of time allowed for dephosphorylation against the desired extent of dephosphorylation for any particular individual assay, to facilitate a rapid yet reliable and accurate identification of inhibitors of HePTP.

Those skilled in the art will also understand how to optimize any given assay in terms of the temperature at which the dephosphorylation reaction is run; however, as a general guide which should be suitable for most, if not all, reactions, depending, once again on the choice of reagents and the desired result, a suitable temperature range would be from about room temperature, about 25° C. to about 37° C., with a temperature closer to room temperature suitable as one example. Those skilled in the art would also know what conventional methods to use to adjust the temperature to within the desired range either before or during the selected period of time for dephosphorylation.

Step (d)

Step (d) quantitating the amount of dephosphorylation in the first cocktail by comparing the amount of dephosphorylation in the first cocktail with the amount of dephosphorylation in a control wherein the control comprises all of the ingredients of said first cocktail except for said test agent (potential HePTP inhibitor). When, as described herein above, a reagent such as Biomol Green™ is used, the amount of phosphate complex can be measured at a wavelength either suggested by the manufacturer or at a wavelength determined by the formulator through experimentation. Alternatively, substrate can be used that provides a measurable parameter. For example, the dephosphorylation of the reagent p-nitrophenyl phosphate gives as one of the products of this reaction, p-nitrophenol. This compound is highly colored and a measurement of the absorbance at a convenient wavelength of light can serve as a measure of the degree of dephosphorylation. One example of a wavelength that is suitable for measuring the amount ofp-nitrophenol present due to the activity of HePTP in the presence of a test compound is 405 nm (nanometers) using a molar extinction coefficient of 18,000 M⁻¹cm⁻¹. Measurement using a PowerWaveX340 microplate spectrophotometer (Bio-Tek Instruments, Inc.) is an example of an instrument suitable for determining the amount ofp-nitrophenol present.

The formulator can use any scalars to measure the amount of desphosphorylation. One example is provided herein above under the heading “Activity Scoring,” however, the formulator can use any criteria to assess whether a compound is a suitable inhibitor of HePTP activity. For example, the formulator can set a minimum difference in absorbance measured at a specific wavelength of light between the first cocktail (compound to be tested) and the second cocktail (control).

Any suitable reaction vessel can be used in the methods of the present invention. The reaction vessel may be of any suitable design, e.g., shape, surface area, volume, and the like, and comprised of any suitable material. Suitable reaction vessels include, for example, microtiter plates, e.g., 48-well or 96-well microtiter plates, e.g., COSTAR #3690 plate or Greiner 384-well clear microtiter plates (781101). In addition, the subject assays can be performed on a desired larger scale, for example, by using an automated, e.g., robotic, system.

The present disclosure further relates to a method for inhibiting HePTP activity comprising contacting HePTP with one or more compounds as disclosed herein. The method can include inhibiting the activity of HePTP in a cell, wherein the inhibition of HePTP in a cell can be done in vivo or ex vivo.

The present disclosure further relates to a method for inhibiting HePTP wherein one or more of the compounds disclosed herein are administered to a patient in need of treatment for a disease affected by HePTP activity.

The present disclosure yet further relates to a method for treating leukemia, myelodysplastic syndrome or acute myelogenous leukemia in a human by inhibiting, reducing, modifying, modulating, or otherwise controlling the activity of HePTP.

### HePTP INHIBITORS

The following compounds are disclosed herein as inhibitors of HePTP that are useful for the treatment of leukemia, pre-leukemic conditions, as well as myelodysplastic syndrome and acute myelogenous leukemia.

One example of HePTP inhibitors disclosed herein are compounds and pharmaceutically acceptable salts thereof having Formula (I):

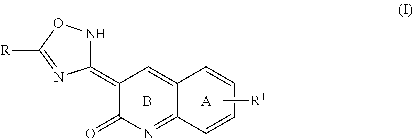

wherein R is phenyl or phenyl substituted by from 1 to 5 organic radicals comprising from 1 to 4 carbon atoms; and R¹ is from 1 to 4 optional organic radical substitutes for hydrogen on the A ring.

One example of compounds having Formula (I) are compounds wherein R^{1a}, R^{1b}, R^{1c}, and R^{1d} are each independently hydrogen or an organic radical comprising from 1 to 4 carbon atoms; R is phenyl or phenyl substituted by from 1 to 5 C₁-C₄ alkyl, C₁-C₄ alkoxy, hydroxy, halogen, amino, monoalkylamino, dialkylamino, carboxy, acyl, or nitro units.

One iteration of this example of compounds having Formula (I) are compounds wherein R is phenyl or phenyl substituted by one or more halogen. Non-limiting examples of R according to this example are chosen from phenyl, 2-fluorophenyl, 2-chlorophenyl, 3-fluorophenyl, 3-chlorophenyl, 4-fluorophenyl, 4-chlorophenyl, 2,3-difluorophenyl, 2,3-dichlorophenyl, 2,4-difluorophenyl, 2,4-dichlorophenyl, 2,5-difluorophenyl, 2,5-dichlorophenyl, 2,6-difluoro-phenyl, 2,6-dichlorophenyl,3,4-difluorophenyl, 3,4-dichlorophenyl, 3,5-difluorophenyl, and 3,5-dichlorophenyl.

Another iteration of this example of compounds having Formula (I) includes compounds wherein R is chosen from 2-hydroxyphenyl, 3-hydroxyphenyl, 4-hydroxyphenyl, 2,3-dihydroxyphenyl, 2,4-dihydroxyphenyl, 2,5-dihydroxyphenyl, 2,6-dihydroxyphenyl, 3,4-dihydroxyphenyl, 3,5-dihydroxyphenyl, 2-methoxyphenyl, 3-methoxyphenyl, 4-methoxyphenyl, 2,3-dimethoxyphenyl, 2,4-dimethoxyphenyl, 2,5-dimethoxyphenyl, 2,6-dimethoxyphenyl, 3,4-dimethoxyphenyl, 3,5-dimethoxyphenyl, 2-ethoxyphenyl, 3-ethoxyphenyl, 4-ethoxyphenyl, 2,3-diethoxyphenyl, 2,4-diethoxyphenyl, 2,5-diethoxyphenyl, 2,6-diethoxyphenyl, 3,4-diethoxyphenyl, and 3,5-diethoxyphenyl.

A further iteration of this example of compounds having Formula (I) includes compounds wherein R is chosen from 2-aminophenyl, 3-aminophenyl, 4-aminophenyl, 2-(methylamino)phenyl, 3-(methylamino-)phenyl, 4-(methylamino)phenyl, 2-(dimethylamino)phenyl, 3-(dimethylamino)phenyl, and 4-(dimethylamino)phenyl.

Another example of compounds having Formula (I) includes compounds having the Formula (Ia):

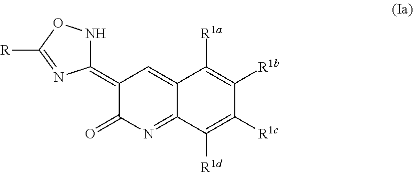

wherein R^{1a}, R^{1b}, R^{1c}, and R^{1d} are each independently chosen from:
a) hydrogen;
b) C₁-C₄ linear, branched, or cyclic alkyl;
c) C₁-C₄ linear, branched, or cyclic alkoxy;
d) C₁-C₄ linear, branched, or cyclic haloalkyl;
e) C₁-C₄ linear, branched, or cyclic haloalkoxy;
f) hydroxy;
g) cyano;
h) nitrilo;
i) nitro;
j) nitroso;
k) amino;
l ) monoalkylamino.
m) dialkylamino;
n) acyl;
o) carboxy;
p) acyloxy;
q1) thioalkyl; and
r) sulfo.
s) sulfoxy; and
t) sulfonamide.

Examples include compounds wherein R^{1a} and R^{1d} are both hydrogen. A further example of Formula (IIa) includes compounds wherein R^{1b} and R¹c are each independently hydrogen, C₁-C₄ alkyl, or C₁-C₄ alkoxy. Embodiments of this example include compounds wherein R^{1c} is hydrogen and R^{1b} is chosen from methyl, hydroxy, methoxy, trifluoromethyl, fluoro, chloro, and nitro. Another embodiment includes compounds wherein R^{1b} is methoxy.

The following Table I provides HePTP inhibition results for non-limiting examples of compounds having Formula (I).

TABLE I

| | IC₅₀ | | % Inhib. |
|---|---|---|---|
| Compound | (μM) | n* | at 20 μM |
| 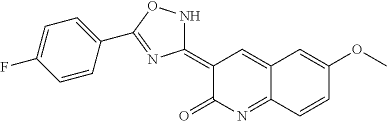 (3Z)-3-[5-(4-fluorophenyl)-1,2,4-oxadiazol-3- ylidene]-6-methoxy-quinolin-2-one | 2.18 | 2.73 | 99.2 |
| 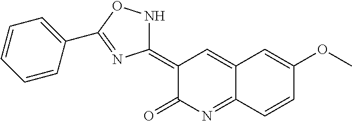 (3Z)-6-methoxy-3-(5-phenyl-1,2,4-oxadiazol-3- ylidene)quinolin-2-one | 4.23 | 4.29 | 77.1 |

* n represents the Hill coefficient. This coefficient is derived from the Hill equation which has the formula:

$$Θ = \frac{{[ L ]}^{n}}{{( {K}_{a} )}^{n} + {[ L ]}^{n}}$$

wherein θ is the fraction of ligand binding sites filled, L is the inhibitor concentration, Kₐ is the inhibitor concentration producing half occupation of the ligand binding sites, and n is the Hill coefficient.

A further example of HePTP inhibitors includes compounds having Formula (II):

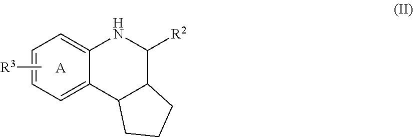

wherein R² is chosen from:
a) —C(O)R⁴;
b) —OC(O)R⁴;
c) —C(O)NR⁵R⁶; and
d) —OC(O)NR⁵R⁶;
   R⁴ is hydrogen, hydroxyl, C₁-C₄ alkyl, and C₁-C₄ alkoxy;
   R⁵ and R⁶ are each independently hydrogen or C₁-C₄ alkyl; or R⁵ and R⁶ can be taken together to form a ring having from 2 to 6 carbon atoms;
   R³ is from 1 to 4 optional organic radical substitutes for hydrogen atoms on the A ring of Formula (II), wherein R³ can comprise from 1 to 10 carbon atoms.

One example of compounds having Formula (II) are compounds wherein R² is chosen from —C(O)R⁴ and —C(O)NR⁵R⁶ wherein R⁴ is hydroxyl or methoxy, R⁵ and R⁶ are each hydrogen or methyl. An embodiment of this example includes compounds having Formula (IIa):

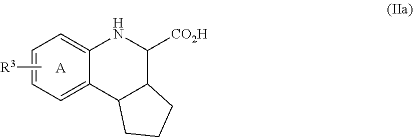

wherein R³ is further defined herein.

A further example of compounds according to Formula (II) are compounds having the Formula (IIa):

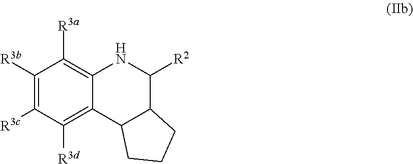

wherein R² is —C(O)OH or —C(O)NH₂; R^{3a}, R^{3b}, R^{3c}, and R^{3d} represent optional substitutions for hydrogen atoms independently chosen from phenyl and substituted phenyl, alkyl, alkoxy, hydroxy, halogen, amino, alkylamino, carboxy (ester), carboxy (amide), and acyl.

An example of compounds according to Formula (Hb) have the formula:

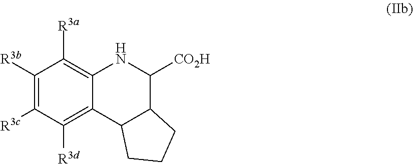

wherein R^{3a}, R^{3b}, R^{3c}, and R^{3d} are each independently chosen from hydrogen, C₁-C₄ alkyl, C₁-C₄ alkoxy, halogen, hydroxy, or —C(O)OR^{3e}, R^{3e} is substituted alkyl, phenyl, or benzyl, the substitutions are chosen from hydroxy, methyl, methoxy, and halogen.

Another example includes compounds of Formula (Hb) wherein R^{3a}, R^{3b}, and R^{3c} are each independently hydrogen, methyl, hydroxyl, or methoxy, and R^{3d} is hydrogen

A further embodiment of this example includes compounds according to Formula (IIb) wherein R^{3a}, R^{3b}, and R^{3c} are each independently hydrogen, methyl, hydroxyl, or methoxy. Further embodiments include compounds wherein R^{3a} and R^{3b} are each methyl and wherein R^{3a} is hydroxy or methoxy. A yet another example includes compounds wherein R^{3c} is phenoxycarbonyl.

The following Table II provides HePTP inhibition results for non-limiting examples of compounds having Formula (II).

TABLE II

| | IC₅₀ | | % Inhib. |
|---|---|---|---|
| Compound | (μM) | nH* | at 20 μM |
| 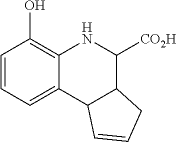 6-hydroxy-3a,4,5,9b-tetrahydro-3H- cyclopenta[c]quinoline-4-carboxylic acid | 0.2 | 2.11 | 103.1 |
| 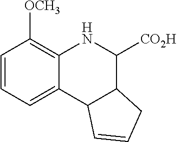 6-methoxy-3a,4,5,9b-tetrahydro-3H- cyclopenta[c]quinoline-4-carboxylic acid | 1.28 | 1.54 | 102.8 |
| 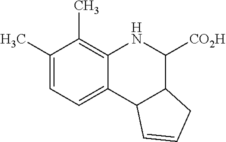 6,7-dimethyl-3a,4,5,9b-tetrahydro-3H- cyclopenta[c]quinoline-4-carboxylic acid | 2.12 | 2.01 | 102.2 |
| 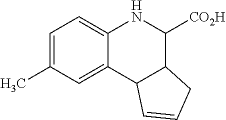 8-methyl-3a,4,5,9b-tetrahydro-3H- cyclopenta[c]quinoline-4-carboxylic acid | 0.41 | 2.41 | 102.1 |
| 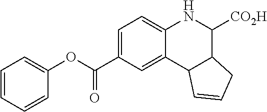 8-(phenoxycarbonyl)-3a,4,5,9b-tetrahydro-3H- cyclopenta[c]quinoline-4-carboxylic acid | 0.84 | 3.34 | 96.4 |
| 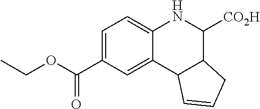 8-(ethoxycarbonyl)-3a,4,5,9b-tetrahydro-3H- cyclopenta[c]quinoline-4-carboxylic acid | 0.28 | 2.97 | 103.3 |

A further example of HePTP inhibitors relates to compounds having Formula (III):

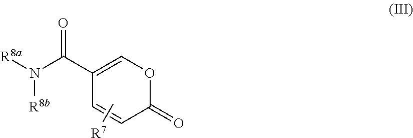

wherein R⁷ is 1 or 2 optional organic radicals that can have from 1 to 4 carbon atoms that are substitutes for hydrogen;

R^{8a} and R^{8b} are taken together to form a ring having from 3 to 7 atoms that can be optionally substituted with from 1 to 6 organic radicals that can have from 1 to 6 carbon atoms and the ring can have from 1 to 3 heteroatoms chosen from nitrogen, oxygen, and sulfur.

One example of compounds according to Formula (III) includes compounds wherein R⁷ is halogen, for example, fluoro, chloro, or bromo.

Another example of compounds according to Formula (III) includes compounds wherein R⁷ is C₁-C₄ alkyl, for example, methyl, ethyl, n-propyl, and iso-propyl.

A further example of compounds according to Formula (III) includes compounds wherein R^{8a} and R^{8b} are taken together form a substituted or unsubstituted ring having from 2 to 7 carbon atoms and from 1 to 3 heteroatoms chosen from nitrogen, oxygen, and sulfur, for example, a 5-member or 6-member nitrogen containing ring optionally comprising one or more nitrogen, oxygen, or sulfur atoms. One embodiment of this example includes compounds wherein R^{8a} and R^{8b} are taken together to form a substituted or unsubstituted ring chosen from piperidinyl, piperazinyl, pyrrolidinyl, pyrrolyl, pyridinyl, pyrimidinyl, and morpholinyl that can be further substituted by alkyl, alkoxy, halogen, and the like. For example, substituted can be methyl, ethyl, methoxy, or fluoro.

The following Table III provides HePTP inhibition results for non-limiting examples of compounds having Formula (III).

TABLE III

| | IC₅₀ | | % Inhib. |
|---|---|---|---|
| Compound | (μM) | nH* | at 20 μM |
| 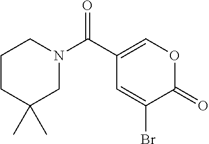 3-bromo-5-(3,3-dimethylpiperidine- 1-carbonyl)pyran-2-one | 0.4 | 2.66 | 101.6 |
| 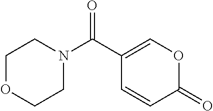 5-morpholine-4-carbonyl)pyran-2-one | 0.99 | 1.02 | 54.1 |

Another example of the HePTP inhibitors disclosed herein includes compounds having Formula (IV):

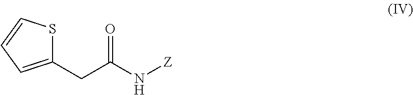

wherein Z is a substituted or unsubstituted 5-member ring heteroaryl unit that can be optionally substituted by from 1 to 4 organic radicals that can have from 3 to 10 carbon atoms. Non-limiting examples of organic radicals that can substitute for hydrogen on a Z unit include organic radicals chosen from alkyl, halogen, phenyl, benzyl and acyl, each of which can be substituted by one or more alkyl, alkoxy, halogen, cyano, nitro, amino, alkylamino, dialkylamino, and thioalkyl. Non-limiting examples of 5-member heteroaryl rings include thiophene, thiazole, isothiazole, 1,3,4-thiadiazole, oxazole, isoxazole, imidazole, and the like.

One example of HePTP inhibitors having formula (IV) includes compounds having Formula (IVa):

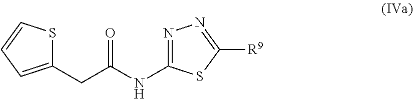

wherein R⁹ is alkyl, halogen, phenyl, benzyl, acyl, all of which can be optionally substituted by one or more organic radicals chosen from alkyl, alkoxy, halogen, cyano, nitro, amino, alkylamino, dialkylamino, and thioalkyl.

The following Table IV provides HePTP inhibition results for non-limiting examples of compounds having Formula (IV).

TABLE IV

| | IC₅₀ | | % Inhib. |
|---|---|---|---|
| Compound | (μM) | nH* | at 20 μM |
| 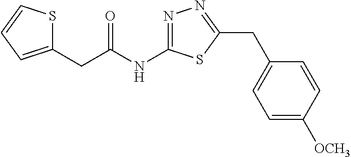 N-{5-[(4-methoxyphenyl)methyl]-1,3,4- thiadiazol-2-yl}-2-thiophen-2-ylacetamide | 4.02 | 2.33 | 102.6 |
| 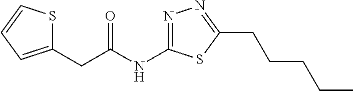 N-(5-pentyl-1,3,4-thiadiazol-2-yl)-2- thiophen-3ylacetamide | 0.49 | 1.86 | 100.5 |
| 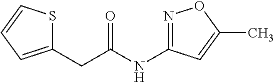 N-(5-methyl-1,2-oxazol-3-yl)-2-thiophen-2-ylacetamide | 0.7 | 2.49 | 99.7 |

Table V provide a list of non-limiting examples of HePTP inhibitors according to the present disclosure

TABLE V

| | IC₅₀ | | % Inhib. |
|---|---|---|---|
| Compound | (μM) | nH* | at 20 μM |
| 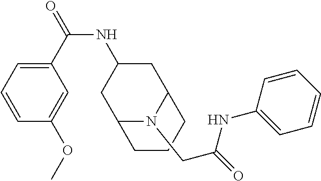 3-methoxy-N-[9-(phenylcarbamoylmethyl)-9- azabicyclo[3.3.1]non-7-yl]benzamide hydrochloride | 4.67 | 4.22 | 102.6 |
| 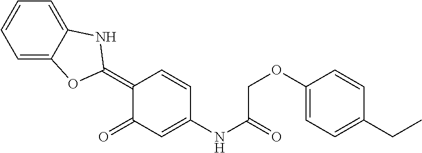 N-[(4Z)-4-(3H-Benzooxazol-2-ylidene)-3-oxo-1- cyclohexa-1,5-dienyl]-2-(4-ethylphenoxy)acetamide | 5.28 | 0.29 | 59 |
| 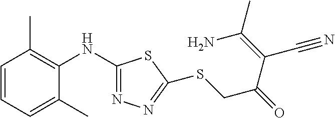 (Z)-3-Amino-2-[2-[[5-[(2,6-dimethylphenyl)amino]- 1,3,4-thiadiazol-2-yl]sulfanyl]acetyl]but-2-enenitrile | 30.2 | 2.66 | 77.9 |
| 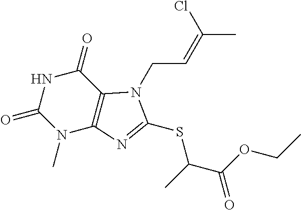 Ethyl 2-[7-[(Z)-3-Chlorobut-2-enyl]-3-methyl-2,6- dioxo-purin-8-yl]sulfanylpropanoate | 8.33 | 1.44 | 55.4 |
| 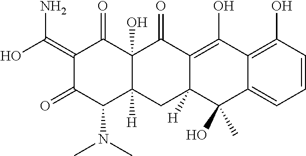 (2E,4S,4aS,5aS,6S,12aS)-2-(Amino-hydroxy- methylidene)-4-dimethylamino-6,10,11,12a-tetrahydroxy- 6-methyl-4,4a,5,5a-tetrahydrotetracene-1,3,12-trione | 8.33 | 4.41 | 98 |
| 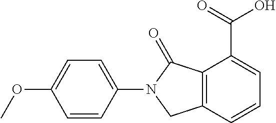 2-(4-Methoxyphenyl)-3-oxo-1H-isoindole-4- carboxylic acid | 1.17 | 3.7 | 57.2 |
| 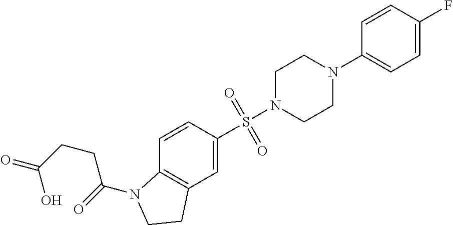 4-[5-[4-(4-Fluorophenyl)piperazin-1-yl]sulfonyl-2,3- dihydroindol-1-yl]-4-oxo-butanoic acid | 3.77 | 7.72 | 58.8 |
| 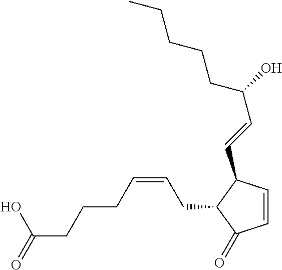 (Z)-7-[(1R,2S)-2-[(E,3S)-3-Hydroxyoct-1-enyl]-5- oxo-1-cyclopent-3-enyl]hept-5-enoic acid | 13.7 | 2.49 | 57.3 |
| 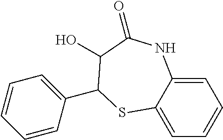 4-Hydroxy-3-phenyl-2-thia-6- azabicyclo[5.4.0]undeca-7,9,11-trien-5-one | 1.15 | 2.13 | 67.2 |
| 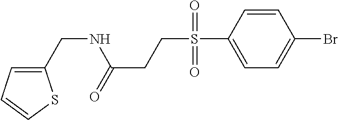 3-(4-Bromophenyl)sulfonyl-N-(thiophen-2- ylmethyl)propanamide | 68.34 | 1.24 | 102.8 |
| 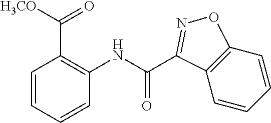 Methyl 2-(4-oxa-3-azabicyclo[3.3.0]octa-2,9-diene-2- carbonylamino)benzoate | 2.33 | 1.45 | 81.2 |
| 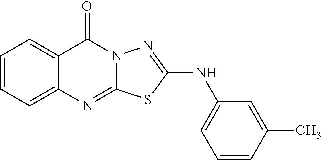 2-(3-Methylphenylamino)-5H-[1,3,4]thiadiazolo[2,3- b]quinazolin-5-one | 2.74 | 1.32 | 53.9 |
| 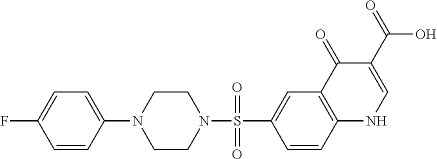 6-[4-(4-Fluorophenyl)piperazin-1-yl]sulfonyl-4-oxo- 1H-quinoline-3-carboxylic acid | 4.66 | 2.66 | 103.2 |
| 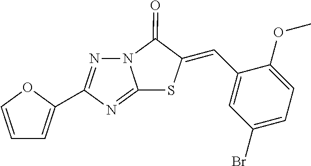 3-[(5-Bromo-2-methoxy-phenyl)methylidene]-7-(2- furyl)-4-thia-1,6,8-triazabicyclo[3.3.0]octa-5,7-dien-2-one | 0.42 | 2.5 | 95.3 |
| 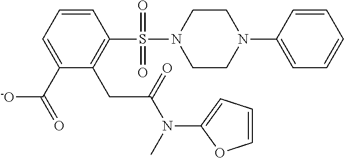 2-Furylmethylcarbamoylmethyl 3-(4-phenylpiperazin- 1-yl)sulfonylbenzoate | 4.97 | 2.07 | 50.7 |
| 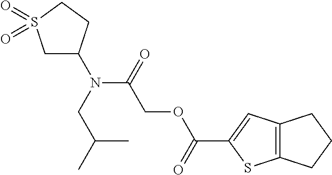 [(1,1-Dioxothiolan-3-yl)-(2-methylpropyl)carbamoyl]methyl 4- thiabicyclo[3.3.0]octa-2,9-diene-3-carboxylate | 1.21 | 1.52 | 58.7 |
| 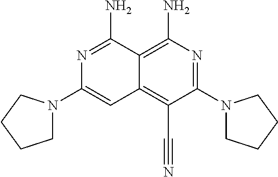 1,8-Diamino-3,6-dipyrrolidin-1-yl-2,7-naphthyridine- 4-carbonitrile | 6.96 | 0.99 | 61.3 |
| 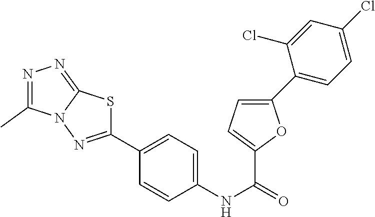 5-(2,4-Dichlorophenyl)-N-[4-(2-methyl-6-thia-1,3,4,8- tetrazabicyclo[3.3.0]octa-2,4,7-trien-7- yl)phenyl]furan-2-carboxamide | 1.5 | 1.94 | 102.2 |
| 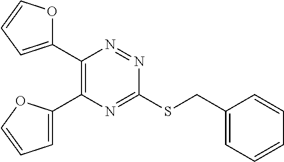 3-Benzylsulfanyl-5,6-bis(2-furyl)-1,2,4-triazine | 2.74 | 1.32 | 53.9 |
| 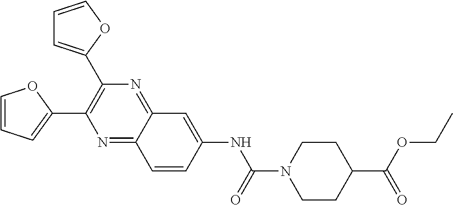 Ethyl 1-[[2,3-bis(2-furyl)quinoxalin-6- yl]carbamoyl]piperidine-4-carboxylate | 1.12 | 1.95 | 103.1 |
| 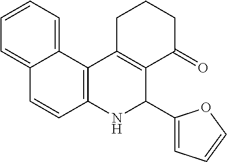 5-(Furan-2-yl)-2,3,5,6- tetrahydrobenzo[a]phenantridin-4(1H)-one | 5.95 | 2.08 | 59.4 |
| 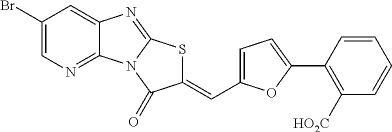 | 1.79 | 7.56 | 102.5 |
| 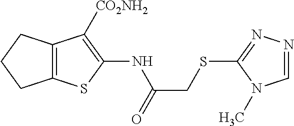 N-[3-(aminooxycarbonyl)-5,6-dihydro-4H- cyclopenta[b]thiophen-2-yl]-2-(4-methyl-4H-1,2,4- triazol-3-ylthio)acetamide | 2.43 | 2.33 | 90.2 |
| 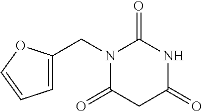 1-(2-Furylmethyl)-1,3-diazinane-2,4,6-trione | 1.31 | 1.67 | 84.9 |
| 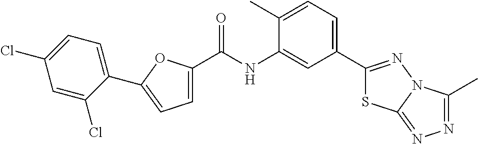 5-(2,4-Dichlorophenyl)-N-[2-methyl-5-(2-methyl-6- thia-1,3,4,8-tetrazabicyclo[3.3.0]octa-2,4,7-trien-7- yl)phenyl]furan-2-carboxamide | 2.12 | 1.3 | 92.4 |
| 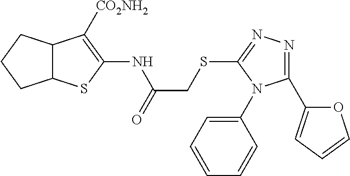 3-[[2-[[5-(2-furyl)-4-phenyl-1,2,4-triazol-3 - yl]sulfanyl]acetyl]amino]-4-thiabicyclo[3.3.0]octa- 2,9-diene-2-carboxamide | 10.6 | 4.06 | 97.5 |
| 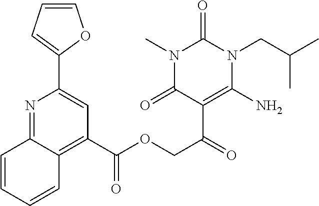 [2-[4-Amino-1-methyl-3-(2-methylpropyl)-2,6-dioxo- pyrimidin-5-yl]-2-oxo-ethyl] 2-(2-furyl)quinoline-4-carboxylate | 5.27 | 2.48 | 89.7 |
| 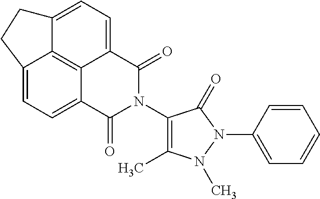 2-(1,5-Dimethyl-3-oxo-2-phenyl-2,3-dihydro-1H- pyrazol-4-yl)-6,7-dihydro-1H-indeno[6,7,1- def]isoquinoline-1,3(2H)-dione | 1.17 | 2.23 | 93.1 |
| 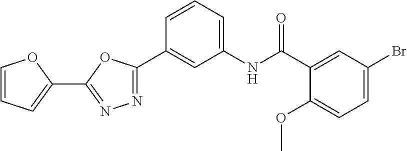 5-Bromo-N-[3-[5-(2-furyl)-1,3,4-oxadiazol-2- yl]phenyl]-2-methoxy-benzamide | 4.9 | 1.47 | 102.5 |
| 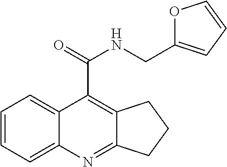 N-(Furan-2-ylmethyl)-2,3-dihydro-1h- cyclopenta[b]quinoline-9-carboxamide; | 4.63 | 1.52 | 62 |
| 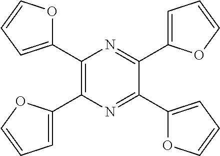 2,3,5,6-tetrakis(2-furyl)pyrazine | 1.14 | 2.26 | 74.1 |
| 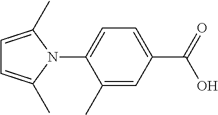 4-(2,5-Dimethylpyrrol-1-yl)-3-methyl-benzoic acid | 0.7 | 2.09 | 103.1 |
| 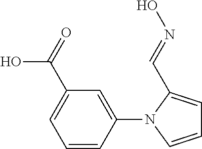 3-[2-(Hydroxyiminomethyl)pyrrol-1-yl]benzoic acid | 1.4 | 1.45 | 58.1 |
| 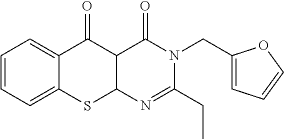 2-Ethyl-3-furan-2-ylmethyl)-3H-thiochromeno[2,3- d]pyrimidine-4,5(4aH,10aH)-dione | 15.09 | 1.27 | 79.4 |
| 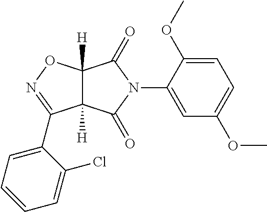 (1S,5S)-2-(2-chlorophenyl)-7-(2,5-dimethoxyphenyl)- 4-oxa-3,7-diazabicyclo[3.3.0]oct-2-ene-6,8-dione | 8.25 | 2.27 | 85.1 |
| 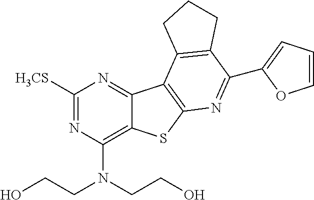 | 0.41 | 2.28 | 62.5 |
| 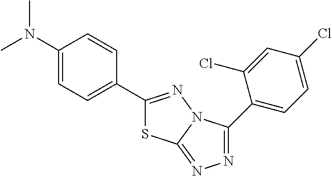 4-[2-(2,4-Dichlorophenyl)-6-thia-1,3,4,8-tetrazabicyclo[3.3.0] octa-2,4,7-trien-7-yl]-N,N-dimethyl-aniline | 78.4 | 2.71 | 64.3 |
| 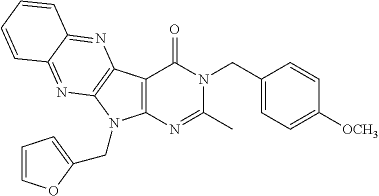 | 6.86 | 2.17 | 103.8 |
| 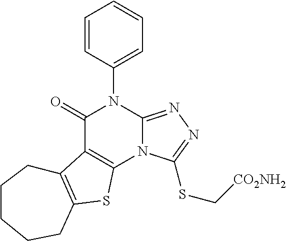 | 2.54 | 2.26 | 83.4 |
| 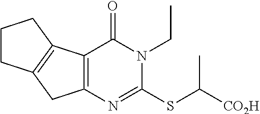 2-(3-Ethyl-4-oxo-3,4,5,6,7,8-hexahydropentaleno[2,1- d][uro,odom-2-ylthio)propanoic acid | 1.69 | 3.77 | 86 |
| 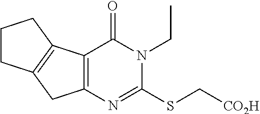 2-(3-Ethyl-4-oxo-3,4,5,6,7,8-hexahydropentaleno[2,1- d][uro,odom-2-ylthio)acetic acid | 1.29 | 4.48 | 102.2 |
| 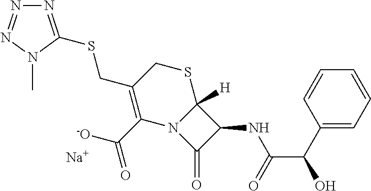 Sodium (6R,7S)-7-[[(2R)-2-hydroxy-2-phenyl- acetyl]amino]-3-[(1-methyltetrazol-5-yl)sulfanylmethyl]- 8-oxo-5-thia-1-azabicyclo[4.2.0]oct-2-ene-2-carboxylate | 0.1 | 0.6 | 98 |
| 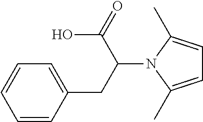 2-(2,5-Dimethylpyrrol-1-yl)-3-phenyl-propanoic acid | 0.42 | 275 | 102.2 |
| 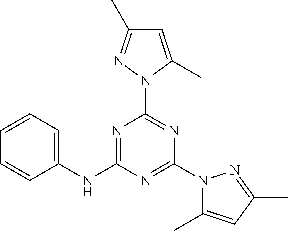 4,6-bis(3,5-Dimethylpyrazol-1-yl)-N-phenyl-1,3,5- triazin-2-amine | 21.6 | 2.83 | 50.5 |
| 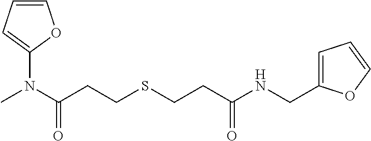 N-(2-Furylmethyl)-3-[2-(2- furylmethylcarbamoyl)ethylsulfanyl]propanamide | 4.15 | 1.32 | 100.6 |
| 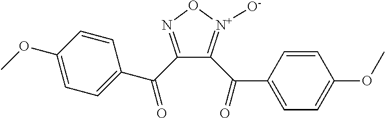 [4-(4-Methoxybenzoyl)-2-oxido-1-oxa-5-aza-2- azoniacyclopenta-2,4-dien-3-yl]-(4-methoxyphenyl)methanone | 11.27 | 0.45 | 86.3 |
| 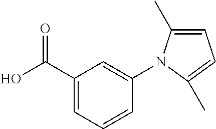 3-(2,5-Dimethylpyrrol-1-yl)benzoic acid | 1.3 | 2.59 | 102 |
| 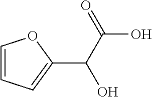 2-(2-Furyl)-2-hydroxy-acetic acid | 2.3 | 1.79 | 100 |
| 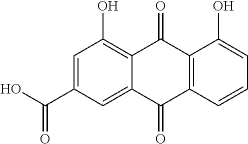 4,5-Dihydroxy-9,10-dioxo-anthracene-2-carboxylic acid | 19.62 | 0.76 | 64.2 |
| 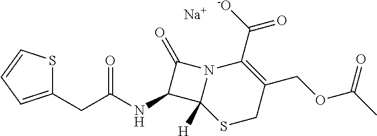 Sodium (6R,7S)-3-(acetyloxymethyl)-8-oxo-7-[(2- thiophen-2-ylacetyl)amino]-5-thia-1- azabicyclo[4.2.0]oct-2-ene-2-carboxylate | 0.92 | 1.12 | 96.2 |

### FORMULATIONS

The present disclosure also relates to compositions or formulations which comprise the HePTP inhibitors according to the present disclosure. In general, the compositions of the present disclosure comprise:
a) an effective amount of one or more HePTP inhibitors according to the present disclosure can be used for treating leukemia, pre-leukemic conditions, myelodysplastic syndrome and acute myelogenous leukemia; and
b) one or more excipients.

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present disclosure have improved cellular potency, pharmacokinetic properties, as well as improved oral bioavailability.

Non-limiting examples of compositions according to the present disclosure include:
a) from about 0.001 mg to about 1000 mg of one or more HePTP inhibitors according to the present disclosure; and
b) one or more excipients.

Another example according to the present disclosure relates to the following compositions:
a) from about 0.01 mg to about 100 mg of one or more HePTP inhibitors according to the present disclosure; and
b) one or more excipients.

A further example according to the present disclosure relates to the following compositions:
a) from about 0.1 mg to about 10 mg of one or more human protein HePTP inhibitors according to the present disclosure; and
b) one or more excipients.

The term “effective amount” as used herein means “an amount of one or more HePTP inhibitors, effective at dosages and for periods of time necessary to achieve the desired or therapeutic result.” An effective amount may vary according to factors known in the art, such as the disease state, age, sex, and weight of the human or animal being treated. Although particular dosage regimes may be described in examples herein, a person skilled in the art would appreciated that the dosage regime may be altered to provide optimum therapeutic response. For example, several divided doses may be administered daily or the dose may be proportionally reduced as indicated by the exigencies of the therapeutic situation. In addition, the compositions of the present disclosure can be administered as frequently as necessary to achieve a therapeutic amount.

As described herein above, the formulations of the present disclosure include pharmaceutical compositions comprising a compound that can inhibit the activity of HePTP and therefore is suitable for use in treating leukemia, pre-leukemic conditions, including myelodysplastic syndrome, and acute myelogeneous leukemia(or a pharmaceutically-acceptable salt thereof) and a pharmaceutically-acceptable carrier, vehicle, or diluent. Those skilled in the art based upon the present description and the nature of any given inhibitor identified by the assays of the present invention will understand how to determine a therapeutically effective dose thereof.

The pharmaceutical compositions may be manufactured using any suitable means, e.g., by means of conventional mixing, dissolving, granulating, dragee-making, levigating, emulsifying, encapsulating, entrapping or lyophilizing processes.

Pharmaceutical compositions for use in accordance with the present disclosure thus may be formulated in a conventional manner using one or more physiologically or pharmaceutically acceptable carriers (vehicles, or diluents) comprising excipients and auxiliaries which facilitate processing of the active compounds into preparations which can be used pharmaceutically. Proper formulation is dependent upon the route of administration chosen.

Any suitable method of administering a pharmaceutical composition to a patient may be used in the methods of treatment of the present invention, including injection, transmucosal, oral, inhalation, ocular, rectal, long acting implantation, liposomes, emulsion, or sustained release means.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. For ocular administration, suspensions in an appropriate saline solution are used as is well known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator, may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, such as sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

One type of pharmaceutical carrier for hydrophobic compounds of the invention is a cosolvent system comprising benzyl alcohol, a nonpolar surfactant, a water-miscible organic polymer, and an aqueous phase.

The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g., polyvinyl pyrrolidone; and other sugars or polysaccharides may be substituted for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed.

Additionally, the compounds may be delivered using any suitable sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a prolonged period of time. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the agents of the invention may be provided as salts with pharmaceutically acceptable counterions. Salts tend to be more soluble in aqueous or other protonic solvents than are the corresponding free base forms.

Other aspects of the present invention include methods of treating a condition or a disease in a mammal comprising administering to said mammal a pharmaceutical composition of the present invention.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

1-44. (canceled)

45. A method of treating leukemia, myelodysplastic syndrome or acute myelogenous leukemia, comprising administering to a patient an effective amount of a compound with the formula 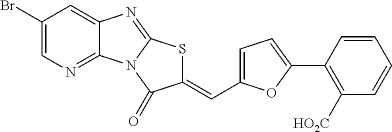
or a pharmaceutically acceptable salt thereof.

46. The method of claim 45, wherein the method treats leukemia.

47. The method of claim 48, wherein the method treats myelodysplastic syndrome.

48. The method of claim 48, wherein the method treats acute myelogenous leukemia.

49. A method for inhibiting HePTP activity comprising contacting HePTP with an effective amount of a compound with the formula 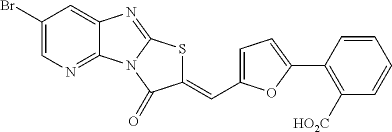
or a pharmaceutically acceptable salt thereof.

50. The method of claim 49, wherein the HePTP activity is in a patient.

51. The method of claim 49, wherein the compound is administered to a patient in need of treatment for a disease affected by HePTP activity.

52. The method of claim 51, wherein the disease is leukemia.

53. The method of claim 51, wherein the disease is myelodysplastic syndrome.

54. The method of claim 51, wherein the disease is acute myelogenous leukemia.

55. The method of claim 49, wherein the activity is inhibited in a cell.

56. The method of claim 55, wherein the activity is in vivo.

57. The method of claim 55, wherein the activity is ex vivo.

58. A composition for treating leukemia, pre-leukemic conditions, myelodysplastic syndrome and acute myelogenous leukemia comprising:
a) an effective amount of a compound with the formula 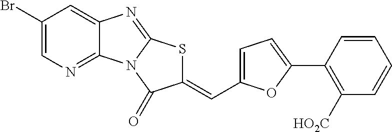
or a pharmaceutically acceptable salt thereof; and
b) one or more excipients.
